(19) United States
(12) Patent Application Publication
(10) Pub. No.: US 2011/0272155 A1
(43) Pub. Date: Nov. 10, 2011

(54) System and method for fluid treatment

(75) Inventor: Wesley J. Warren, Duncan, OK (US)

(73) Assignee: HALLIBURTON ENERGY SERVICES, INC., Houston, TX (US)

(21) Appl. No.: 12/774,393

(22) Filed: May 5, 2010

(51) Int. Cl. E21B 43/12; A61L 2/10

(52) U.S. Cl. 166/275; 422/24; 250/436

(57) ABSTRACT

A method of treating a wellbore servicing fluid includes disposing an ultraviolet treatment chamber in fluid communication with an inlet of a pump, operating the pump, and drawing a wellbore servicing fluid through the ultraviolet treatment chamber in response to operating the pump. A wellbore servicing fluid treatment system has a first ultraviolet treatment chamber and a first pump in selective fluid communication with the first ultraviolet treatment chamber. The pump is downstream relative to the first ultraviolet treatment chamber and the pump is configured to selectively draw a wellbore servicing fluid through the first ultraviolet treatment chamber. A method of servicing a wellbore includes connecting an ultraviolet fluid treatment system to a blender, operating a pump of the blender, drawing a wellbore servicing fluid through the ultraviolet treatment system in response to operating the pump of the blender, and delivering the wellbore servicing fluid into the wellbore.

### CROSS-REFERENCE TO RELATED APPLICATIONS

None.

### STATEMENT REGARDING FEDERALLY SPONSORED RESEARCH OR DEVELOPMENT

Not applicable.

### REFERENCE TO A MICROFICHE APPENDIX

Not applicable.

### FIELD OF THE INVENTION

This invention relates to systems and methods of treating fluids associated with servicing wellbores.

### BACKGROUND OF THE INVENTION

Suitable fluid supplies are sometimes required to perform wellbore servicing operations and to produce wellbore servicing fluids. However, a fluid supply may be abundant but nonetheless unusable and/or undesirable due to the presence of bacteria, non-beneficial microorganisms, and/or other undesirable organic compositions of the fluid supply. Some systems utilize ultraviolet (UV) radiation to improve usability of the fluids for wellbore servicing operations and for producing wellbore servicing fluids. Some UV fluid treatment systems require a pump upstream from the UV treatment chamber, such as a centrifugal pump, to deliver fluid to the UV fluid treatment system. The use of an upstream pump for delivering fluid to the UV fluid treatment system is costly in terms of equipment and labor. Further, the rate of fluid delivery from the upstream pump to the UV fluid treatment system sometimes does not substantially match the rate at which the treated fluid from the UV fluid treatment system is used and/or demanded. Because the UV treated fluid may be provided at rates dissimilar from the rate at which UV treated fluid is used and/or demanded, it is sometimes further necessary to store the UV treated fluid in a fluid store. A result of storing the treated fluid in a fluid store, such as a frac tank or other fluid storage means, may be that the UV treated fluid is degraded in response to undesirable mixing with bacteria, non-beneficial microorganisms, and/or other undesirable organic compositions. Further, because the rate of fluid delivery from the high pressure pump to the UV treatment system may be inconsistent, the UV treated fluid may be treated at different dosage rates of UV radiation. Additionally, the fluid rate must be controlled to ensure the tank is not overfilled or emptied. Accordingly, there is a need for a UV fluid treatment system and method that does not require an upstream pump to supply fluid to the UV fluid treatment and/or does not require storage of treated fluid in fluid stores prior to use of the UV treated fluid.

### SUMMARY OF THE INVENTION

In some embodiments, a method of treating a wellbore servicing fluid is disclosed. The method may comprise disposing an ultraviolet treatment chamber in fluid communication with an inlet of a pump, operating the pump, and drawing a wellbore servicing fluid through the ultraviolet treatment chamber in response to operating the pump.

In other embodiments, a wellbore servicing fluid treatment system having a first ultraviolet treatment chamber and a first pump in selective fluid communication with the first ultraviolet treatment chamber is disclosed. The pump may be downstream relative to the first ultraviolet treatment chamber and the pump may be configured to selectively draw a wellbore servicing fluid through the first ultraviolet treatment chamber.

In yet other embodiments, a method of servicing a wellbore is disclosed. The method may comprise transporting an ultraviolet fluid treatment system to a location near the wellbore, connecting the ultraviolet fluid treatment system to a blender, operating a pump of the blender, drawing a wellbore servicing fluid through the ultraviolet treatment system in response to operating the pump of the blender, and delivering the wellbore servicing fluid into the wellbore.

### BRIEF DESCRIPTION OF THE DRAWINGS

FIG. 1 is a simplified schematic view of an ultraviolet fluid treatment system according to an embodiment of the disclosure;

FIG. 2 is a simplified partial oblique top view of the ultraviolet fluid treatment system of FIG. 1;

FIG. 3 is another simplified schematic view of the ultraviolet fluid treatment system of FIG. 1 shown as including an air removal system;

FIG. 4 is a simplified side view of the ultraviolet fluid treatment system in a transport position;

FIG. 5 is a simplified side view of the ultraviolet fluid treatment system in an operating position;

FIG. 6 is another simplified schematic view of the ultraviolet fluid treatment system in a circulation configuration;

FIG. 7 is a simplified side view of a UV treatment chamber of the ultraviolet fluid treatment system of FIG. 1; and

FIG. 8 is a simplified schematic view of a wellbore servicing system according to an embodiment of the disclosure.

### DETAILED DESCRIPTION OF THE PREFERRED EMBODIMENTS

In the drawings and description that follow, like parts are typically marked throughout the specification and drawings with the same reference numerals, respectively. The drawing figures are not necessarily to scale. Certain features of the invention may be shown exaggerated in scale or in somewhat schematic form and some details of conventional elements may not be shown in the interest of clarity and conciseness.

Disclosed herein are systems and methods for treating a fluid with UV radiation and for using the UV treated fluid in wellbore servicing operations and/or using the UV treated fluid in the production of wellbore servicing fluids. A greater understanding of some of the problems posed by the presence of organic contaminants in fluids for use in wellbore servicing operations may be found in U.S. Pat. No. 7,332,094 which is hereby incorporated by reference in its entirety. Some of the systems and methods disclosed herein are directed toward providing a UV fluid treatment system (hereinafter referred to as “UVFTS”) that does not require the use of an upstream pump (e.g., an upstream pump that may produce about 10 psi to about 50 psi) to provide fluid to the UVFTS. In particular, some of the systems and methods disclosed herein are directed toward providing a UVFTS that may be provided fluid and/or primed as a result of the gravitational potential energy of a store of the untreated fluid. Further, some of the systems and methods disclosed herein are directed toward providing a UVFTS that is provided fluid as a result of a pressure differential generated by a pump downstream of the UVFTS and/or where the UVFTS is provided fluid through a fluid conduit that is not highly pressurized. In other words, in some embodiments, the pressure in the UVFTS may be substantially equal to the head pressure of a frac tank minus the friction pressure attributable to the piping connections between the frac tank and the UVFTS. In some embodiments, depending upon the tank head pressure and the pressure loss attributable to the piping system, a pressure in the UVFTS may be less than atmospheric pressure. Still further, some of the systems and methods disclosed herein are directed toward providing a UVFTS having decreased susceptibility to failure as a result of air accumulation within the UVFTS.

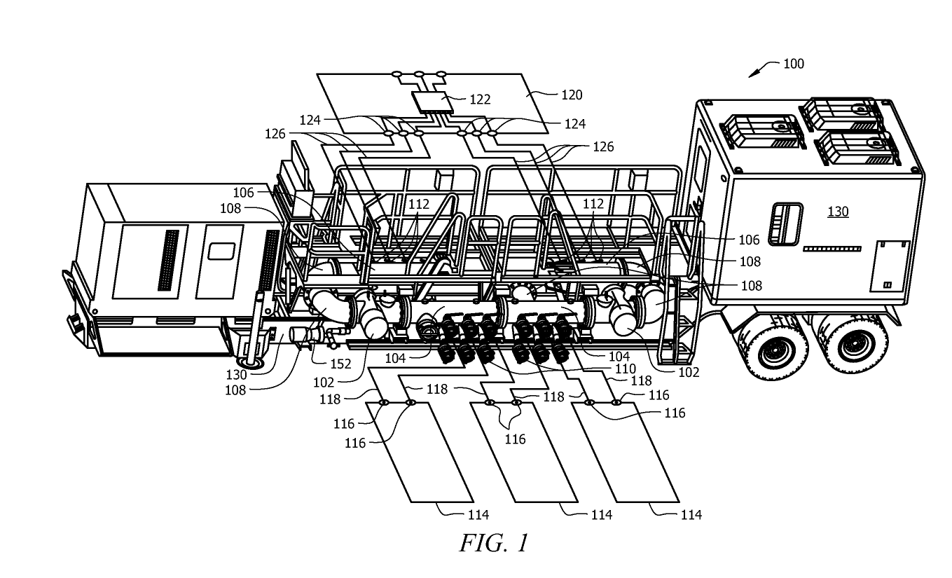

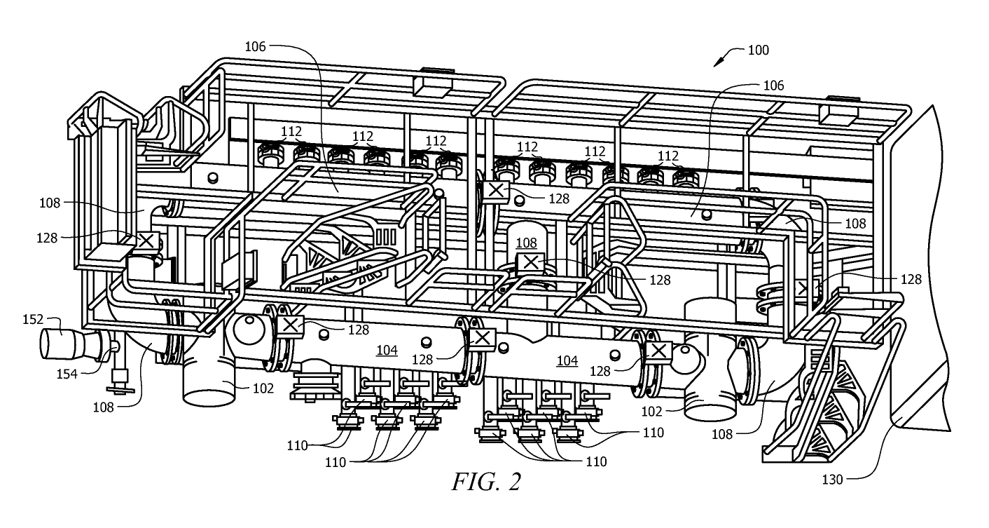

FIGS. 1 and 2 are simplified views of a UVFTS 100 according to an embodiment. FIG. 1 shows UVFTS 100 as connected to additional wellbore servicing equipment while FIG. 2 provides a more detailed view of the UVFTS 100 itself. UVFTS 100 generally comprises UV treatment chambers 102 in selective fluid connection with input headers 104. The input headers 104 are further connected to output headers 106 by intermediate pipes 108. The input headers 104 generally comprise a plurality of input ports 110 for receiving fluid into the input headers 104. The output headers 106 generally comprise a plurality of output ports 112 through which fluid (untreated, treated, and/or a combination of treated and untreated fluid) may flow out of the UVFTS 100. UVFTS 100 is shown as connected to a plurality of fluid sources 114 (i.e., frac tanks), each fluid source 114 comprising one or more fluid source outlets 116. The fluid source outlets 116 may be connected to input ports 110 of the UVFTS 100 by fluid conduits 118. UVFTS 100 is further shown as connected to a blender 120 that comprises a pump 122. The output ports 112 of UVFTS 100 may be connected to blender input ports 124 via additional fluid conduits 126.

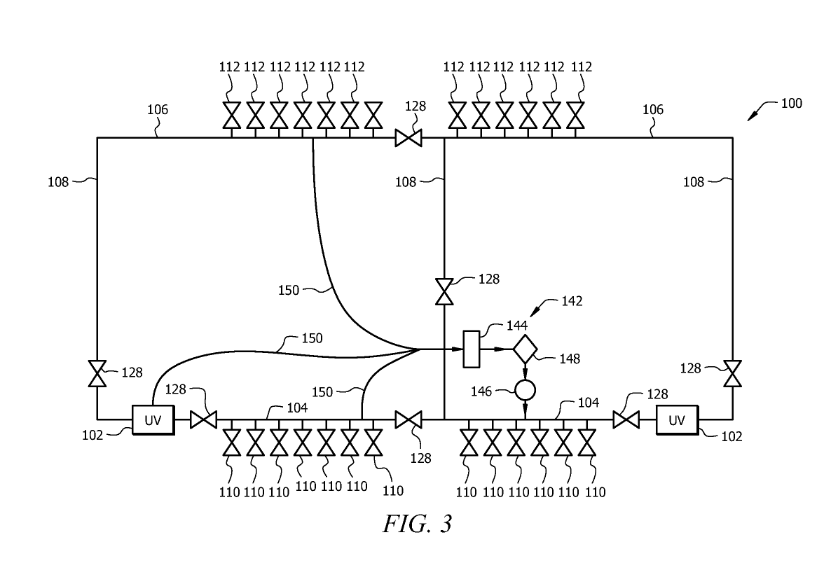

Referring additionally to FIG. 3 which provides a simplified schematic view of UVFTS 100, in this embodiment, the UVFTS 100 comprises two UV treatment chambers 102. However, in alternative embodiments, a UVFTS 100 may comprise more or fewer chambers 102. In this embodiment, the chambers 102 are most generally connected in parallel between the input headers 104 and the output headers 106 so that fluid may flow between the input headers 104 and the output headers 106 via one or both chambers 102. A plurality of valves 128 are provided to selectively allow and/or restrict fluid flow through the various input headers 104, output headers 106, and intermediate pipes 108. Accordingly, fluid may be selectively directed through any of a variety of paths between the input ports 110 and the output ports 112.

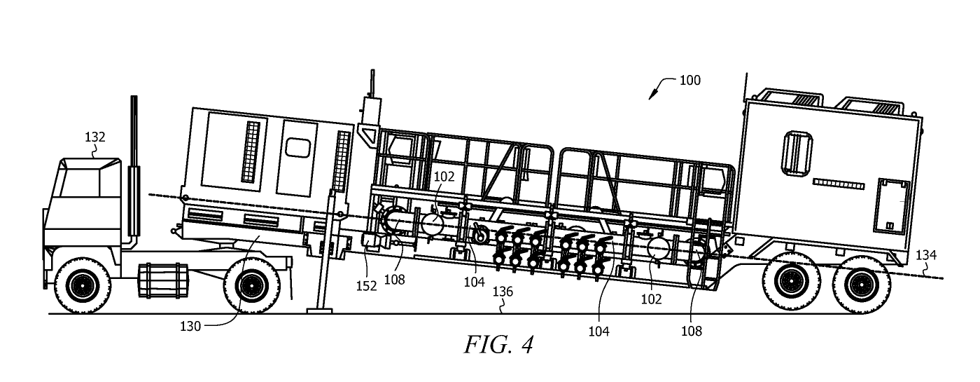

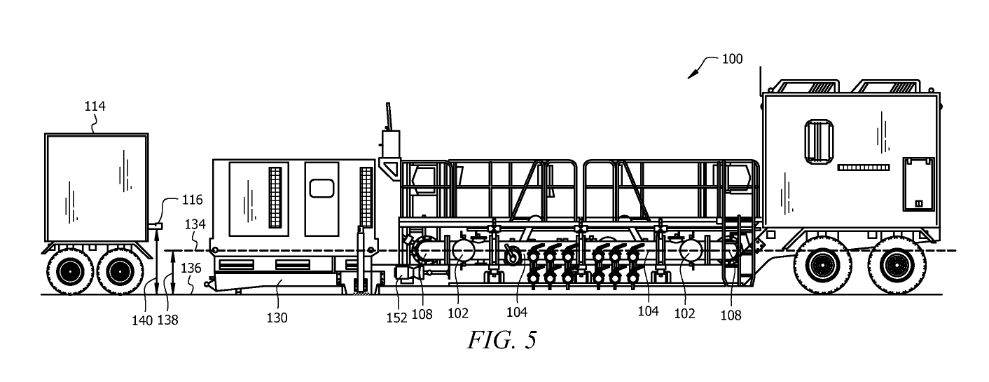

Referring now to FIGS. 4 and 5, the UVFTS 100 is more clearly shown as installed and/or carried on a trailer 130 for transport and/or delivery to and from locations near wellbores by a tractor 132. In particular, FIG. 4 shows the UVFTS 100 installed on the trailer 130 and further shows the UVFTS 100 and the trailer 130 in a transport position. When the UVFTS 100 is in a transport position, a generally centralized plane 134 of the UVFTS 100 is incident to level ground 136. On the other hand, FIG. 5 shows the UVFTS 100 and the trailer 130 in an operating position where the centralized plane 134 of the UVFTS 100 is substantially parallel to level ground 136. The centralized plane 134, in this embodiment, may be generalized as being a plane that substantially bisects the vertical heights of one or more of the chambers 102, input headers 104, output headers 106, and intermediate pipes 108. In this embodiment, the centralized plane 134 substantially bisects the vertical heights of each of the chambers 102, input headers 104, output headers 106, and intermediate pipes 108. Most generally, the centralized plane 134 may be offset from level ground 136 by a plane offset distance 138 when the UVFTS 100 and the trailer 130 are in the operating position.

Also shown in FIG. 5 is a fluid store 114 (i.e., a frac tank) having a fluid source outlet 116. The fluid source outlet 116 may be offset from level ground 136 a greater distance than the plane offset distance 138. In this embodiment, a lower portion of the fluid source outlet 116 is located an outlet offset distance 140 above level ground 136 by an amount so that at least a portion of the fluid source outlet 116 is located vertically higher than at least a portion of at least one of the chambers 102, input headers 104, output headers 106, and intermediate pipes 108. Due to this relative height difference between the fluid source outlet 116 and the components of the UVFTS 100, a fluid head pressure differential may exist between the fluid source outlet 116 and the input ports 110. In such cases, fluid may flow through fluid conduits 118 from the fluid source outlet 116 to the input ports 110 primarily as a result of gravitational potential energy of the wellbore servicing fluid. In some embodiments, even though the outlet offset distance 140 may be less than the height of the plane offset distance 138, fluid may nonetheless flow from the fluid store 114 to the UVFTS 100 due to the gravitational potential energy as long as the fluid level in the fluid store 114 is above a plane of at least a portion of at least one of the chambers 102, input headers 104, output headers 106, and intermediate pipes 108.

Referring back to FIG. 3, the UVFTS 100 may be provided with an air removal system 142. The air removal system may comprise a relatively low flow rate air removal pump 144 that draws fluid (i.e., trapped air and/or wellbore servicing fluid) from vertically high spaces of one or more of the chambers 102, input headers 104, output headers 106, and intermediate pipes 108. In some embodiments, the low flow rate of the air removal pump 144 may be about 10 gallons per minute while a flow rate through the UVFTS 100 during operation of the UVFTS 100 may be significantly higher, such as, but not limited to, about 100 barrels per minute or any other suitable flow rate for providing UV treated fluid. The air removal pump 144 may pump fluid through a check valve 146 and through an air vent valve 148 to remove excess and/or accumulated air pockets from the UVFTS 100. Air removal lines 150 illustrate only a few of the possible locations for drawing fluid from the UVFTS 100. The air removal system 142 allows air to escape the UVFTS 100 while returning the other fluid (primarily liquid) to the input headers 104. It will be appreciated that the air vent valve 148, in some embodiments, may need to be placed in a pressurized air removal line 150 to operate properly. Accordingly, in some embodiments where the pressure within the UVFTS 100 may be low and/or lower than atmospheric pressure, the air vent valve 148 may not be well suited for installation directly within one or more of the chambers 102, input headers 104, output headers 106, and intermediate pipes 108 of the UVFTS 100. Further, in some embodiments, the check valve 146 may comprise a biasing member and/or spring that disallows opening of the check of the check valve 146 until a force from the fluid pressure acting on the check exceeds the biasing force of the member and/or spring of the check valve 146. Accordingly, in some embodiments, the above-described check valve 146 functionality may provide a pressurized air removal line 150 between the air removal pump 144 and the check valve 146 where the air vent valve 148 may be installed.

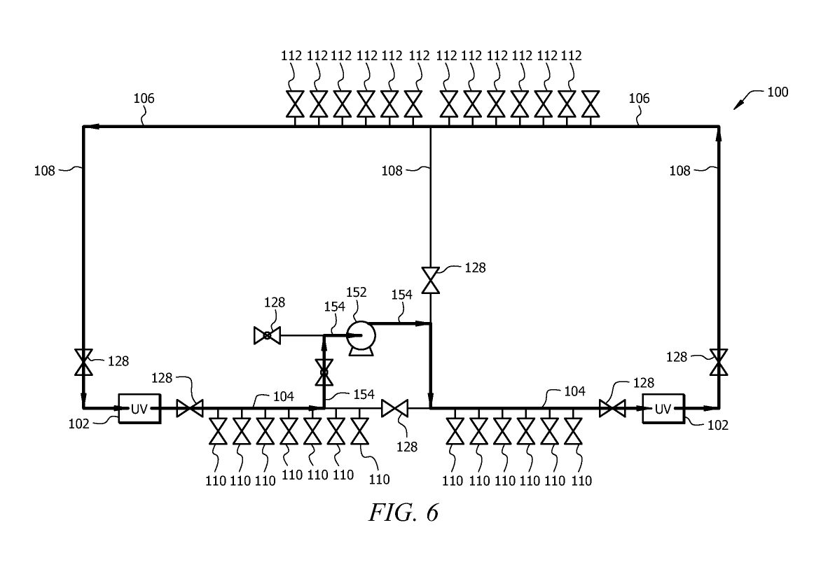

Referring now to FIG. 6, another schematic view of the UVFTS 100 is shown. FIG. 6 illustrates that the UVFTS 100 may be provided with a circulating pump 152 and appropriate fluid conduits 154 for circulating fluids through the chambers 102 during startup (i.e., warm-up of chambers 102), shutdown (i.e., cool down of chambers 102), and/or during any other interruption of fluid flow into and/or out of the UVFTS 100. FIG. 6 shows that the valves 128 may be used to prevent fluid flow through one or more intermediate pipes 108 to provide predictable serial fluid flow through the multiple chambers 102. In other words, the valves 128 may be used to disable the parallel fluid flow between the input headers 104 and the output headers 106. Each input port 110 and each output port 112 may comprise integral and/or associated valves 128 to further enable the fluid circulation shown.

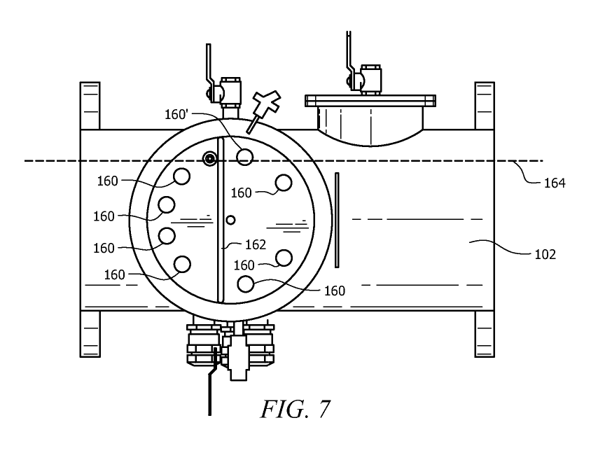

Referring now to FIG. 7, a UV treatment chamber 102 is shown as comprising a plurality of UV radiation emitting devices 160. In this embodiment, the devices 160 are electrically powered UV lamps that may require a period of operation prior to reaching full and/or desirable radiation output levels. However, in alternative embodiments, any other suitable on-demand source of ultraviolet radiation may be used as a device 160. The plurality of devices 160 may be substantially rod-like in shape and may extended generally transversely into and orthogonal to a lengthwise central axis of the generally cylindrical UV treatment chamber 102. Accordingly, it can be seen that some devices 160 are located vertically higher than other devices 160 within the same chamber 102. Because some devices 160 may malfunction or overheat when operated without being sufficiently submerged in fluids, control systems may be provided that disable insufficiently submerged devices 160, throttle down power usage of insufficiently submerged devices 160, stepwise reduce UV emissions of insufficiently submerged devices 160, and/or alter a pulse width type modulation of insufficiently submerged devices 160. For example, FIG. 7 depicts an insufficiently submerged device 160′. A sensor 162 (i.e., temperature and/or fluid level sensor) may be used to determine which of the devices 160 may be insufficiently submerged because a portion of the device 160′ extends above a fluid level 164. In alternative embodiments, a sensor 162 may be placed in any other component of UVFTS 100 (i.e., input headers 104, output headers 106, and/or intermediate pipes 108) so long as a meaningful relationship between the fluid levels of those components is related to the fluid levels within one or more chambers 102. In some embodiments, each chamber 102 may comprise a dedicated sensor 162 for sensing a fluid level and contributing to the control of the devices 160. In alternative embodiments, a single sensor 162 may contribute to the control of devices 160 of more than one chamber 102.

In operation of some embodiments, a UVFTS 100 may be transported to a location near a wellbore to be treated. Once located near the wellbore to be treated, the UVFTS 100 may be altered from a transportation position to an operation position in which the central plane 134 is caused to be generally parallel to level ground 136. Further, with the UVFTS 100 in the operation position, at least a portion of a fluid level in the fluid source 114 may be vertically higher than a portion of at least one of the chambers 102, inlet headers 104, outlet headers 106, and intermediate pipes 108. With such relative height differences, fluid may freely flow as assisted by gravity to at least partially fill one or more of the components of the UVFTS 100, thereby priming the UVFTS 100.

Once the UVFTS 100 is primed, valves 128 and circulating pump 152 may be utilized to create a circulating fluid circuit that directs wellbore servicing fluid through the chambers 102 as the chambers 102 are warmed up or otherwise powered up to a desired operating capacity. When the devices 160 are ready to provide desired UV radiation output, circulating pump 152 may be disabled and/or selectively isolated from the UVFTS 100 and the valves 128 may be actuated and/or otherwise configured to allow fluid passage from the inlet ports 110 to the outlet ports 112. In other words, upon operation of the pump 122 of the blender 120, fluid may be drawn (via suction pressure generated by pump 122) from the fluid sources 114, through the UVFTS 100, and into the blender 120. In the blender 120, wellbore servicing fluid additive constituents may be added to the fluid to generate a wellbore servicing fluid and/or mixture for delivery downhole into the wellbore (i.e., delivery downhole for fracturing or other wellbore services) and/or for storage for later use.

As disclosed above, the UVFTS 100 may provide the benefit of an ultraviolet fluid treatment system that does not require an upstream pump (e.g., a centrifugal pump operable at about 50 psi or less) to deliver fluid to the UVFTS 100. As explained above, the UVFTS 100 of the present invention is operable without such an upstream pump, in some embodiments, because the fluid sources 114 comprise fluid levels that are at least partially vertically higher than at least a portion of the fluid components of the UVFTS 100. The UVFTS 100 described above further allows treatment of fluid at rates substantially similar to the rates needed by the blender 120. Because the fluid transferred through the UVFTS 100 is primarily accomplished in response to operation of pump 122 of blender 120, UV dosage rates can be controlled and/or known and treated fluid need not be exposed to fluid contaminants normally encountered in treated fluid storage devices. The parallel fluid circuit formed between the inlet headers 104 and the outlet headers 106 also reduces fluid restriction and/or pressure loss across the UVFTS 100 (as compared to the fluid restriction and/or pressure loss across serially connected chambers 102), thereby permitting the pump 122 to easily draw fluid through the UVFTS 100.

While the UVFTS 100 has been described as comprising two chambers 102, in alternative embodiments, a UVFTS may comprise more than two chambers 102 and may comprise backup and/or redundancy chambers 102 in case of failure of one or more primary chambers 102. In some embodiments, the UVFTS 100 may eliminate a need for separate flow meters and management of flow meters for a blender 120 and a UVFTS 100. Instead, in some embodiments, the flow rate though the blender 120 may be substantially equal to the fluid flow rate through the UVFTS 100. In some embodiments, the fluid flow rate through the UVFTS 100 may be substantially similar to the rate at which fluid is delivered downhole in a wellbore, for example, during a wellbore fracturing operation.

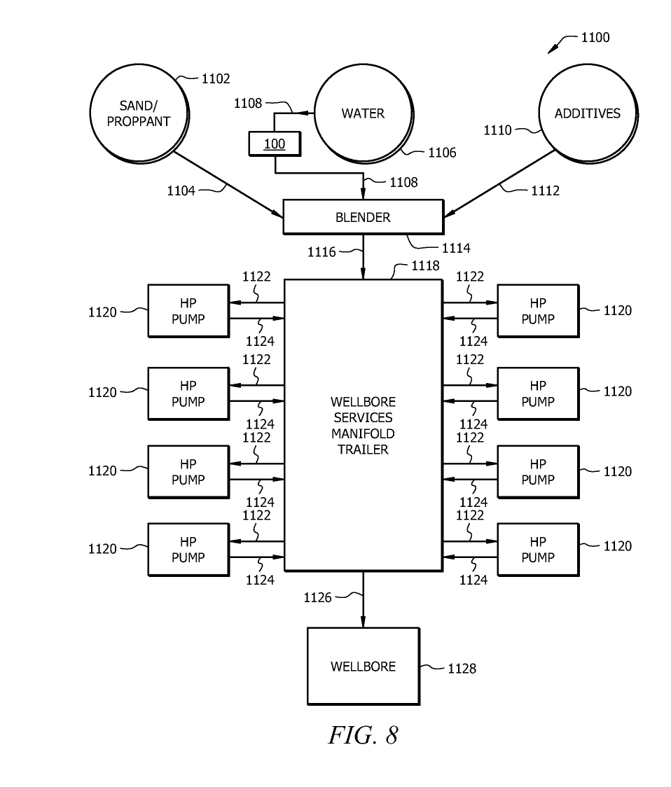

Referring to FIG. 8, a wellbore servicing system 1100 is shown as comprising an embodiment of a UVFTS 100. The wellbore servicing system 1100 is a system for fracturing wells in a hydrocarbon reservoir. In fracturing operations, wellbore servicing fluids, such as particle laden fluids, are pumped at high-pressure into a wellbore. The particle laden fluids may then be introduced into a portion of a subterranean formation at a sufficient pressure and velocity to cut a casing and/or create perforation tunnels and fractures within the subterranean formation. Proppants, such as grains of sand, are mixed with the wellbore servicing fluid to keep the fractures open so that hydrocarbons may be produced from the subterranean formation and flow into the wellbore. Hydraulic fracturing may desirably create high-conductivity fluid communication between the wellbore and the subterranean formation.

The wellbore servicing system 1100 comprises a blender 1114 that is coupled to a wellbore services manifold trailer 1118 via flowline 1116. As used herein, the term “wellbore services manifold trailer” includes a truck and/or trailer comprising one or more manifolds for receiving, organizing, and/or distributing wellbore servicing fluids during wellbore servicing operations. In this embodiment, the wellbore services manifold trailer 1118 is coupled to eight high pressure (HP) pumps 1120 via outlet flowlines 1122 and inlet flowlines 1124. In alternative embodiments, however, there may be more or fewer HP pumps used in a wellbore servicing operation. Outlet flowlines 1122 are outlet lines from the wellbore services manifold trailer 1118 that supply fluid to the HP pumps 1120. Inlet flowlines 1124 are inlet lines from the HP pumps 1120 that supply fluid to the wellbore services manifold trailer 1118.

The blender 1114 mixes solid and fluid components to achieve a well-blended wellbore servicing fluid. As depicted, sand or proppant 1102, water 1106, and additives 1110 are fed into the blender 1114 via feedlines 1104, 1108, and 1112, respectively. The water 1106 may be potable, non-potable, untreated, partially treated, or treated water. In an embodiment, the water 1106 may be produced water that has been extracted from the wellbore while producing hydrocarbons form the wellbore. The produced water may comprise dissolved and/or entrained organic materials, salts, minerals, paraffins, aromatics, resins, asphaltenes, and/or other natural or synthetic constituents that are displaced from a hydrocarbon formation during the production of the hydrocarbons. In an embodiment, the water 1106 may be flowback water that has previously been introduced into the wellbore during wellbore servicing operation. The flowback water may comprise some hydrocarbons, gelling agents, friction reducers, surfactants and/or remnants of wellbore servicing fluids previously introduced into the wellbore during wellbore servicing operations.

The water 1106 may further comprise local surface water contained in natural and/or manmade water features (such as ditches, ponds, rivers, lakes, oceans, etc.). Further, the water 1106 may comprise water obtained from water wells. Still further, the water 1106 may comprise water stored in local or remote containers. The water 1106 may be water that originated from near the wellbore and/or may be water that has been transported to an area near the wellbore from any distance. In some embodiments, the water 1106 may comprise any combination of produced water, flowback water, local surface water, and/or container stored water.

In this embodiment, the blender 1114 is an Advanced Dry Polymer (ADP) blender and the additives 1110 are dry blended and dry fed into the blender 1114. In alternative embodiments, however, additives may be pre-blended with water using a GEL PRO blender, which is a commercially available preblender trailer from Halliburton Energy Services, Inc., to form a liquid gel concentrate that may be fed into the blender 1114. The mixing conditions of the blender 1114, including time period, agitation method, pressure, and temperature of the blender 1114, may be chosen by one of ordinary skill in the art with the aid of this disclosure to produce a homogeneous blend having a desirable composition, density, and viscosity. In alternative embodiments, however, sand or proppant, water, and additives may be premixed and/or stored in a storage tank before entering a wellbore services manifold trailer 1118. In an embodiment, the water 1106 may be treated via the UVFTS 100 as described in more detail herein.

The HP pumps 1120 pressurize the wellbore servicing fluid to a pressure suitable for delivery into the wellhead 1128. For example, the HP pumps 1120 may increase the pressure of the wellbore servicing fluid to a pressure of up to about 20,000 psi or higher. The HP pumps 1120 may comprise any suitable type of high pressure pump, such as positive displacement pumps.

From the HP pumps 1120, the wellbore servicing fluid may reenter the wellbore services manifold trailer 1118 via inlet flowlines 1124 and be combined so that the wellbore servicing fluid may have a total fluid flow rate that exits from the wellbore services manifold trailer 1118 through flowline 1126 to the flow connector wellbore 1128 of between about 1 BPM to about 200 BPM, alternatively from between about 50 BPM to about 150 BPM, alternatively about 100 BPM. Persons of ordinary skill in the art with the aid of this disclosure will appreciate that the flowlines described herein are piping that are connected together for example via flanges, collars, welds, etc. These flowlines may include various configurations of pipe tees, elbows, and the like. These flowlines connect together the various wellbore servicing fluid process equipment described herein.

In this embodiment, the wellbore servicing system 1100 further comprises a UVFTS 100 of the type described above. The UVFTS 100 is integrated into the wellbore servicing system 1100 in a fluid circuit between the supply of water 1106 and the blender 1114. As such, the UVFTS 100 is configured to accept fluids from supply of water 1106 and selectively treat the fluids as the fluids pass through the UVFTS 100. In this embodiment, the water storage container may comprise fluids from any number of water sources such as water produced from wellbores (produced water), surface water, or potable water. Accordingly, FIG. 8 and the description above clearly illustrate use of the UVFTS 100 in the context of a wellbore servicing operation, and more particularly, the use of the UVFTS 100 in the context of a wellbore fracturing operation.

At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R=Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present invention. The discussion of a reference in the disclosure is not an admission that it is prior art, especially any reference that has a publication date after the priority date of this application. The disclosure of all patents, patent applications, and publications cited in the disclosure are hereby incorporated by reference in their entireties.

What we claim as our invention is:

1. A method of treating a wellbore servicing fluid, comprising:
disposing an ultraviolet treatment chamber in fluid communication with an inlet of a pump;
operating the pump; and
drawing a wellbore servicing fluid through the ultraviolet treatment chamber in response to operating the pump.

2. The method of claim 1, further comprising:
selectively irradiating the wellbore servicing fluid with ultraviolet radiation.

3. The method of claim 2, wherein the irradiating is varied in response to a measured level of wellbore servicing fluid within the ultraviolet fluid treatment chamber.

4. The method of claim 2, wherein the irradiating is varied in response to a measured temperature within the ultraviolet treatment chamber

5. The method of claim 1, further comprising:
providing the wellbore servicing fluid to the ultraviolet treatment chamber in response to a head differential.

6. The method of claim 5, further comprising:
providing the wellbore servicing fluid to the ultraviolet treatment chamber prior to operating the pump.

7. The method of claim 1, further comprising transferring the wellbore servicing fluid through the ultraviolet treatment chamber at a rate substantially defined by a rate of operation of the pump.

8. A wellbore servicing fluid treatment system, comprising:
a first ultraviolet treatment chamber; and
a first pump in selective fluid communication with the first ultraviolet treatment chamber;
wherein the pump is downstream relative to the first ultraviolet treatment chamber and wherein the pump is configured to selectively draw a wellbore servicing fluid through the first ultraviolet treatment chamber.

9. The wellbore servicing fluid treatment system of claim 8, further comprising:
a fluid source having a fluid source outlet in fluid communication with the first ultraviolet treatment chamber;
wherein at least a portion of the first ultraviolet treatment chamber is located relatively lower than at least a portion of the fluid source level.

10. The wellbore servicing fluid treatment system of claim 9, wherein the fluid source is a frac tank.

11. The wellbore servicing fluid treatment system of claim 9, further comprising:
an input header;
an output header; and
a second ultraviolet treatment chamber;
wherein the first ultraviolet treatment chamber and the second ultraviolet treatment chamber each provide substantially separate fluid flow paths between the input header and the output header.

12. The wellbore servicing fluid treatment system of claim 11, wherein the separate fluid flow paths are configured to provide parallel flow paths between input header and the output header.

13. The wellbore servicing fluid treatment system of claim 11, further comprising:
an air removal system configured to reduce air accumulation in at least one of the first ultraviolet treatment chamber, the second ultraviolet treatment chamber, the input header, and the output header.

14. The wellbore servicing fluid treatment system of claim 13, the air removal system comprising:
an air removal pump configured to draw air from at least one of the first ultraviolet fluid treatment chamber, the second ultraviolet fluid treatment chamber, the input header, and the output header.

15. The wellbore servicing fluid treatment system of claim 10, wherein the first pump is a pump of a blender.

16. The wellbore servicing fluid treatment system of claim 8, further comprising:
an ultraviolet radiation emitting device; and
a fluid level sensor;
wherein operation of the ultraviolet radiation emitting device is variable in response to information sensed by the fluid level sensor.

17. The wellbore servicing fluid treatment system of claim 8, further comprising:
an ultraviolet radiation emitting device; and
a temperature sensor;
wherein operation of the ultraviolet radiation emitting device is variable in response to information sensed by the temperature sensor.

18. A method of servicing a wellbore, comprising:
transporting an ultraviolet fluid treatment system to a location near the wellbore;
connecting the ultraviolet fluid treatment system to a blender;
operating a pump of the blender;
drawing a wellbore servicing fluid through the ultraviolet treatment system in response to operating the pump of the blender; and
delivering the wellbore servicing fluid into the wellbore.

19. The method of claim 18, further comprising:
selectively irradiating the wellbore servicing fluid with ultraviolet radiation.

20. The method of claim 19, further comprising:
selectively blending the wellbore servicing fluid with another wellbore servicing material.
